(19) United States
(12) Patent Application Publication
(10) Pub. No.: US 2022/0378606 A1
(43) Pub. Date: Dec. 1, 2022

(54) Method and Apparatus to Slow Blood Flow Through Extremities During Chemotherapy

(71) Applicant: POLAR PRODUCTS, INC., Stow, OH (US)

(72) Inventor: William S. Graessle, Hudson, OH (US)

(73) Assignee: POLAR PRODUCTS, INC.

(21) Appl. No.: 17/329,526

(22) Filed: May 25, 2021

(51) Int. Cl. A61F 7/02; A61F 7/00

(52) U.S. Cl. CPC A61F 7/02; A61F 2007/0029; A61F 2007/0056; A61F 2007/0287; A61F 2007/0039

(57) ABSTRACT

A system for minimizing chemotherapy-induced neuropathy in extremities employs coolant maintained within a container in communication with a plurality of extremity coverings having serpentine conduits therein and which receive the coolant from the container at one end of the serpentine conduit and return it to the container from the other end. A pump is interposed in conduits between the coolant container and the extremity coverings to cause the flow of coolant. A control system regulates both the duration of operation of the pump and the coolant flow rate to effectively establish the temperature at the extremity coverings by allowing the coolant a dwell time within the extremity coverings.

### TECHNICAL FIELD

The invention herein resides in the art of medical devices, systems and methods. Particularly, the invention relates to treatments provided to extremities, and most particularly to the hands, feet, fingers and toes of patients. More specifically, the invention relates to a method and apparatus for the treatment of such extremities in response to anecdotal evidence that suggests that cooling the hands and feet during chemotherapy treatments will, in many patients, minimize the impact of neuropathy induced by chemotherapy treatments.

### BACKGROUND OF THE INVENTION

“Neuropathy” is defined by www.Dictionary.com as “damage, disease, or dysfunction of one or more nerves especially of the peripheral nervous system that is typically marked by burning or shooting pain, numbness, tingling, or muscle weakness or atrophy, is often degenerative, and is usually caused by injury, infection, disease, drugs, toxins, or vitamin deficiency.” For purposes herein, neuropathy is considered to be the nerve damage outside of the brain and spinal cord that causes weakness, numbness, and pain where the body is so afflicted, often in body extremities such as the hands, fingers, feet and toes. It may be the result of a range of conditions from diabetes to medical treatments such as chemotherapy.

Of particular interest here is chemotherapy-induced neuropathy. During chemotherapy, the chemicals and drugs employed are carried by the bloodstream to malignant cells and tissue, or to the causative agents of disease, such as viruses, bacteria, or other microorganisms. Chemotherapy is a commonly known treatment for cancer.

In the past, neuropathy has been addressed during chemotherapy with regard to the patient's head and attendant hair loss. However, attention to the extremities, such as hands, feet, fingers and toes, has heretofore been absent, notwithstanding the fact that neuropathy in these extremities has been problematic and well known for years.

There is a need in the art for a method and apparatus for addressing body extremities in an effort to reduce the occurrence of neuropathy therein during chemotherapy. There is a further need in the art for such a method and apparatus that is both efficient and cost effective in use. The art is further in need of a method and apparatus to guard against or minimize neuropathy in body extremities that can be regulated so as to be adaptable to patients of various ages and health conditions. Additionally, there is a need in the art for a method and apparatus that can be regulated to be adaptable to the specific treatment being given to a patient.

### SUMMARY OF INVENTION

In light of the foregoing, it is a first aspect of the invention to provide a method and apparatus for addressing neuropathy in body extremities.

It is another aspect of the invention to provide a method and apparatus for addressing neuropathy in body extremities that is portable, easy to set up and use, efficient and cost effective, employing an apparatus that is self-contained.

Still a further aspect of the invention is to provide a method and apparatus for lowering temperature in body extremities that is adaptable to the patient, considering the age and physical condition of the patient, as well as the treatment being given.

### DISCLOSURE OF INVENTION

The foregoing and other aspects of the invention that will become apparent as the detailed description proceeds are achieved by apparatus for lowering a temperature of body extremities to slow blood flow therethrough during chemotherapy, comprising: a housing; a liquid container within said housing; a plurality of extremity coverings having liquid conduits therein; a plurality of tubes intercommunicating between said liquid conduits of said extremity coverings and said liquid container; a pump interposed between said liquid container and plurality of tubes; and means for controlling passage of liquid from said liquid container, through said extremity coverings, and returned to said liquid container.

Yet further aspects of the invention that will become apparent herein are achieved by a system for lowering a temperature of body extremities to slow blood flow therethrough during chemotherapy, comprising: a housing having a container of coolant therein; a first plurality of extremity coverings having serpentine liquid conduits therein; a second plurality of conduits operatively interconnecting said coolant container and said extremity coverings, said second plurality of conduits accommodating flow of coolant from said coolant container to said extremity coverings and returning said coolant from said extremity coverings to said coolant container; a pump interposed between said coolant container and conduits of said second plurality of conduits accommodating flow of coolant to said extremity coverings; and a control system regulating said flow of coolant.

### DESCRIPTION OF DRAWINGS

For a complete understanding of the various aspects of the invention, reference should be made to the following detailed description and accompanying drawings, wherein:

FIG. 1 is a schematic illustration of a system for cooling extremities during chemotherapy according to the invention; and

FIG. 2 is a detailed schematic of the system within the housing 12 of FIG. 1.

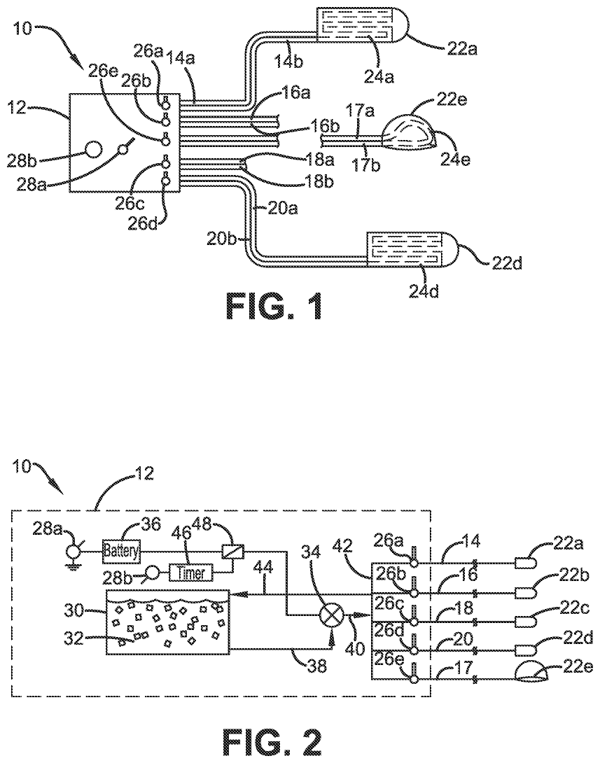

### DETAILED DESCRIPTION OF PREFERRED EMBODIMENT

Referring now to the drawings, it can be seen that a system for slowing blood flow in extremities during chemotherapy is designated generally by the numeral 10. The controls and operative structure of the system 10 are maintained in a housing 12 from which tubes 14-20 extend. Each of the tubes 14-20 comprises a tube pair, each pair having an output tube and an input tube. The output tubes are further designated by the letter “a” and the input tubes by the letter “b.” The tubes 14a,b-20a,b communicate between the housing 12 and hand, fingers, feet, toes and head coverings 22a-22e, each tube pair being uniquely associated with a particular covering. It will be appreciated by those skilled in the art that the tube pairs may be twin pairs connected in side-by-side relationship, or may be coaxial pairs. Each of the coverings 22a-22e is characterized by uniquely associated serpentine tubes 24a-24e. The serpentine tubes of the coverings have a first end interconnected with an associated output tube 14a-20a and a second end interconnected with the input tube 14b-20b. With the “a” tubes and “b” tubes interconnected to opposite ends of the serpentine tubes 24a-e, a path is established between the housing 12, each of the coverings 22a-22e, and back to the housing 12, as will be discussed further below. The coverings 22a-22e are configured to adapt to the part of the body being covered. Typically, the complement of coverings will include a pair of mittens and a pair of socks 22a-d and a head cap 24e.

Valves 26a-26e are interposed within each tube pair 14-20, either the “a” or “b” line, or both, to open and close those respective tubes. A switch 28a is provided to activate the system 10 and particularly a pump as described below. A regulator or control dial 28b is provided to regulate temperature, again in a manner to be discussed below.

As specifically shown in FIG. 2, maintained within the housing 12 is an ice bucket 30, containing ice water 32. As will be appreciated by those skilled in the art, until all of the ice of the ice water 32 is melted, the ice water is nominally maintained at 32° F. Those skilled in the art will appreciate that a powered chiller as well as other coolants may also be used.

A pump 34, operatively powered by a battery 36, or other suitable power source, such as a DC outlet from an automobile, adapter, or the like, draws ice water or coolant 32 from the ice bucket 30 through a tube or other suitable conduit 38. The pump outlet 40 interconnects with a matrix or manifold 42, which in turn is controlled by valves 26a-26e. These valves are positioned within the output tubes 14a-20a, respectively, and enable or inhibit ice water flow to respective coverings 22a-22e. Exiting the coverings 22a-22e, the input tubes 14b-20b receive the ice water or coolant after passing through the coverings and convey that water or coolant through the matrix or manifold 42 to a return line 44, returning the water or coolant to the ice bucket 30.

As shown in FIG. 2, the power source 36 may be activated or disabled by the switch 28a to deliver voltage through the switch 48 to activate the pump 34. The nature of operation of the pump 34 may be controlled by a timer 46 and the associated regulator or control dial 28b. In a first embodiment of the invention, the pump 34 runs continuously, and circulates water or coolant continuously. The timer 46 is simply set by the regulator 28b for a fixed period of time, and the valve 48 remains open for that period of time. The switch 28a may be used to terminate operation at any time as a safety factor or convenience.

According to an alternate embodiment of the invention, if it is desired that the extremity coverings 22 be at a temperature above 32° F., the timer 46 may be set by the regulator 28b to control the valve 48 such that the motor 34 is energized and deactivated at a selected duty cycle. This basically reduces the flow rate of the water and/or increases the dwell time of the water within the coverings 32, such that the heat absorbed from the extremities of the body is reduced with respect to time. Accordingly, without the need for direct temperature regulation, such regulation can be achieved by the implementation of the timer 46 for setting the duty cycle of operation of the pump 34.

The invention also contemplates other means of regulating flow rate, and hence cooling, such as by the use of regulator valves or the like. These valves may be remotely or manually controlled. Indeed, the valves 26a-26e may be simply on/off valves in a basic embodiment of the invention or may be adjustable by the user to control the flow rate therethrough and hence the cooling effect. They may be adjusted independently or they could be connected such that the feet and hands are adjusted in separate pairs, and the head covering alone.

In operation, the ice bucket 30 is filled with ice water 32, and the extremity coverings 22, with the serpentine conduits 24, are placed on selected extremities of the patient. A period of treatment may then be selected by the timer 46. In some embodiments, effective flow rates and cooling effect may be adjusted by either selection of a duty cycle or adjustment of valves 26a-26e.

The system is activated by the switch 28a and the pump 34 operates either continuously or at a preselected duty cycle set by means of the regulator 28b, or at selected flow rates as controlled by valves 26a-26e. The particular extremities of the patient to be cooled during chemotherapy are selected by valves 26a-26e. This cooling contracts the blood vessels, reducing blood flow, and thus, it is believed, reduces the chemotherapy-induced neuropathy at the extremity. The temperature may be regulated to accommodate the age, health anomalies, and other conditions of the patient to ensure that the extremities have sufficient blood flow so as not to result in other damage, while reducing the blood flow in an attempt to preclude or limit neuropathy.

Thus it can be seen that the various aspects of the invention have been satisfied by the structures and methods presented above. While in accordance with the patent statutes only the best known and preferred embodiment of the invention has been presented and described in detail, the invention is not limited thereto or thereby. Accordingly, for an appreciation of the true scope and breadth of the invention reference should be made to the accompanying claims.

What is claimed is:

1. Apparatus for lowering a temperature of body extremities to slow blood flow therethrough during chemotherapy, comprising:
a housing;
a liquid container within said housing;
a plurality of extremity coverings having liquid conduits therein;
a plurality of tubes intercommunicating between said liquid conduits of said extremity coverings and said liquid container;
a pump interposed between said liquid container and plurality of tubes; and
means for controlling operation of said pump to regulate passage of liquid from said liquid container, through said extremity coverings, and returned to said liquid container.

2. The apparatus for lowering a temperature of body extremities to slow blood flow therethrough during chemotherapy as recited in claim 1, wherein said liquid conduits within said extremity coverings pass in a serpentine path within said extremity coverings from an inlet to said extremity coverings from said pump to an outlet from said extremity coverings to said liquid container.

3. The apparatus for lowering a temperature of body extremities to slow blood flow therethrough during chemotherapy as recited in claim 2, wherein said means for controlling passage of liquid comprises valves interposed within at least certain of said plurality of tubes.

4. The apparatus for lowering a temperature of body extremities to slow blood flow therethrough during chemotherapy as recited in claim 3, wherein said valves are adjustable to regulate a flow rate of said liquid through said extremity coverings, thereby regulating a temperature of said liquid in said extremity coverings.

5. The apparatus for lowering a temperature of body extremities to slow blood flow therethrough during chemotherapy as recited in claim 4, wherein said valves are both adjustable and adapted to be fully closed for selecting ones of said extremity coverings for receiving liquid.

6. The apparatus for lowering a temperature of body extremities to slow blood flow therethrough during chemotherapy as recited in claim 4, wherein said liquid container is configured to contain ice water.

7. The apparatus for lowering a temperature of body extremities to slow blood flow therethrough during chemotherapy as recited in claim 6, wherein said extremity coverings comprise mittens, socks and a cap adapted to be placed on extremities of a user for lowering a temperature of said extremities and thereby slowing blood flow through the extremities by cooling the extremities.

8. The apparatus for lowering a temperature of body extremities to slow blood flow therethrough during chemotherapy as recited in claim 5, wherein said means for controlling operation of said pump comprises a power source interconnected through a switch to said pump and an adjustable timer interconnected with said switch, said timer setting a duration of operation of said pump and a duty cycle of such operation.

9. The apparatus for lowering a temperature of body extremities to slow blood flow therethrough during chemotherapy as recited in claim 6, wherein said means for controlling passage of liquid selects between continuous operation of said pump and a settable duty cycle of operation of said pump.

10. The apparatus for lowering a temperature of body extremities to slow blood flow therethrough during chemotherapy as recited in claim 9, wherein said duty cycle of operation of said pump establishes a dwell time of aid ice water within said serpentine paths within said extremity coverings.

11. The apparatus for lowering a temperature of body extremities to slow blood flow therethrough during chemotherapy as recited in claim 10, wherein said dwell time establishes an effective flow rate of said ice water within said serpentine paths within said extremity coverings.

12. A system for lowering a temperature of body extremities to slow blood flow therethrough during chemotherapy, comprising:
a housing having a container of coolant therein;
a first plurality of extremity coverings having serpentine liquid conduits therein;
a second plurality of conduits operatively interconnecting said coolant container and said extremity coverings, said second plurality of conduits accommodating flow of coolant from said coolant container to said extremity coverings and returning said coolant from said extremity coverings to said coolant container;
a pump interposed between said coolant container and conduits of said second plurality of conduits accommodating flow of coolant to said extremity coverings; and
a control system regulating said flow of coolant.

13. The system for lowering a temperature of body extremities to slow blood flow therethrough during chemotherapy according to claim 12, wherein said control system comprises a power source and a timer.

14. The system for lowering a temperature of body extremities to slow blood flow therethrough during chemotherapy according to claim 13, wherein said control system comprises valves within at least certain of said second plurality of liquid conduits.

15. The system for lowering a temperature of body extremities to slow blood flow therethrough during chemotherapy according to claim 14, wherein said valves are both on/off and adjustable to regulate a flow rate of said coolant through said extremity coverings, thereby regulating a temperature of said liquid within said extremity coverings.

16. The system for lowering a temperature of body extremities to slow blood flow therethrough during chemotherapy according to claim 13, wherein said timer effects operation of said pump from the group of a time period of operation and a duty cycle of operation.

17. The system for lowering a temperature of body extremities to slow blood flow therethrough during chemotherapy according to claim 16, wherein said time period, duty cycle, and extremity coverings to which coolant may flow are operator-selectable.
